(19) United States
(12) Patent Application Publication
(10) Pub. No.: US 2013/0172411 A1
(43) Pub. Date: Jul. 4, 2013

(54) STABLE PHARMACEUTICAL COMPOSITIONS COMPRISING FESOTERODINE

(71) Applicants: Sunilendu Bhushan Roy, Gujarat (IN); Sushrut Krishnaji Kulkarni, Gujarat (IN); Pranav Jogani Dhirajbhai, Gujarat (IN); Ajay Handa Kumar, Gujarat (IN)

(72) Inventors: Sunilendu Bhushan Roy, Gujarat (IN); Sushrut Krishnaji Kulkarni, Gujarat (IN); Pranav Jogani Dhirajbhai, Gujarat (IN); Ajay Handa Kumar, Gujarat (IN)

(73) Assignee: CADILA HEALTHCARE LIMITED, Ahmedabad (IN)

(21) Appl. No.: 13/635,977

(22) PCT Filed: Mar. 22, 2011
(86) PCT No.: PCT/IN2011/000189

§ 371 Date: Nov. 19, 2012

(30) Foreign Application Priority Data
Mar. 22, 2010 (IN) 770/MUM/2010

(51) Int. Cl. A61K 31/222

(52) U.S. Cl. CPC A61K 31/222
USPC 514/546

(57) ABSTRACT

The present invention relates to stable pharmaceutical compositions comprising fesoterodine or salts thereof. In particular, the invention relates to pharmaceutical compositions of fesoterodine which does not contain sugar alcohols. The invention also relates to processes for making such compositions and use thereof in treating patients with urinary incontinence.

### FIELD OF THE INVENTION

The present invention relates to stable pharmaceutical compositions comprising fesoterodine or salts thereof. In particular, the invention relates to pharmaceutical compositions of fesoterodine which does not contain sugar alcohols. The invention also relates to processes for making such compositions and use thereof in treating patients with urinary incontinence.

### BACKGROUND OF THE INVENTION

Fesoterodine is a competitive muscarinic receptor antagonist useful in the treatment of overactive bladder with symptoms of urge urinary incontinence, urgency, and frequency. The extended release tablets of fesoterodine fumarate are marketed in USA under the brand name Toviaz® by Pfizer in the strengths of 4 and 8 mg.

Chemically, fesoterodine is 2-[(1R)-3-(diisopropylamino)-1-phenylpropyl]-4-(hydroxymethyl) phenyl isobutyrate.

U.S. Pat. Nos. 6,858,650 and 7,384,980 disclose fesoterodine and its pharmaceutical acceptable salts.

International (PCT) Application No. 2009/044278 discloses amorphous form of fesoterodine fumarate, its process of preparing and pharmaceutical compositions thereof.

U.S. Pat. No. 7,807,715 and U.S. Application Publication No. 2009/117159 disclose a pharmaceutical granulate comprising fesoterodine or a pharmaceutically acceptable salt or solvate thereof and a pharmaceutically acceptable stabilizer, which can be selected from the group consisting of sorbitol, xylitol, polydextrose, isomalt, dextrose, and combinations thereof. According to these applications, sugar alcohols are very essential to prepare stable compositions of fesoterodine.

Fesoterodine is known in the art for its potency in treating urinary incontinence. However, fesoterodine may exhibit substantial degradation under stress conditions, e.g., in a humid environment and, at increased temperature. It is believed that hydrolyzation and oxidation are among the major mechanisms resulting in degradation. Therefore, it would be desirable to develop new pharmaceutical compositions comprising fesoterodine that are more stable against degradation over an extended period of time even under stress conditions.

Prior art attempts as mentioned above exemplify the use of sugar alcohols for improving the stability of fesoterodine compositions. It has been mentioned that sugar alcohols especially xylitol and sorbitol are necessary to make stable compositions of fesoterodine, without which stability is difficult to achieve.

Hence, there remains a need to devise alternative pharmaceutical formulations of fesoterodine without using extra load of excipients, which exhibits significant stability against degradation over the storage period.

### SUMMARY OF THE INVENTION

In one general aspect, there is provided a stable pharmaceutical composition comprising fesoterodine or salts thereof, characterized in that said composition is free of sugar alcohol.

In another general aspect, there is provided a stable pharmaceutical composition comprising fesoterodine or salts thereof, wherein the composition retains at least 80% of potency of fesoterodine or salts thereof when stored at 40° C./75% R.H for 3 months, characterized in that said composition is free of sugar alcohol.

In another general aspect, there is provided a stable pharmaceutical composition comprising fesoterodine or salts thereof, wherein the composition retains at least 80% of potency of fesoterodine or salts thereof when stored at 50° C./80% R.H for at least 1 month, characterized in that said composition is free of sugar alcohol.

In another general aspect, the stable pharmaceutical composition comprising fesoterodine or salts thereof and at least one is rate-controlling polymer, characterized in that said composition is free of sugar alcohol.

In another general aspect, there is provided a pharmaceutical composition comprising fesoterodine or salts thereof suitable for once daily administration, characterized in that said composition is free of sugar alcohol.

Embodiments of the pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, glidants and the like.

In another general aspect, there is provided a process for preparing a stable pharmaceutical composition of fesoterodine or salts thereof, characterized in that said composition is free of sugar alcohol.

In another general aspect, there is provided a process for preparing a stable pharmaceutical composition of fesoterodine or salts thereof comprises of mixing and compressing fesoterodine or salts thereof with one or more pharmaceutically acceptable excipients other than sugar alcohols.

In another general aspect, there is provided a process for preparing a stable pharmaceutical composition of fesoterodine or salts thereof, which process comprises of mixing fesoterodine or salts thereof with one or more rate-controlling polymers, optionally with other pharmaceutically acceptable excipients other than sugar alcohols and converting the mixture thus obtained into suitable pharmaceutical dosage form.

Embodiments of the pharmaceutical composition may include one or more of the following features. For example, the pharmaceutical composition may further include one or more pharmaceutically acceptable excipients. The pharmaceutically acceptable excipients may include one or more binders, fillers, lubricants, disintegrants, glidants and the like.

In another general aspect, there is provided a method of treating a patient suffering from overactive bladder by administering a therapeutically effective amount of a stable pharmaceutical composition of fesoterodine or salts thereof, characterized in that said composition is free of any sugar alcohol.

The details of one or more embodiments of the invention are set forth in the description below. Other features, objects and advantages of the invention will be apparent from the description.

### DETAILED DESCRIPTION OF THE INVENTION

The inventors of the present invention have surprisingly found that it is possible to formulate stable formulations of fesoterodine which overcome all the aforesaid problems without employing any sugar alcohol.

In particular, the inventors have found that when fesoterodine compositions are prepared using a judicial combination of excipients, without using sugar alcohol(s), fesoterodine or its salt exhibits no degradation under stress conditions e.g. humidity or increased temperature rendering the resulting composition stable over the storage period without using extra load of excipients.

Thus, the invention provides a pharmaceutical composition of fesoterodine or salt thereof which remains stable even without employing any sugar alcohol.

The term “Fesoterodine” includes active metabolites or pharmaceutically acceptable solvates of 2-[(1R)-3-(diisopropylamino)-1-phenylpropyl]-4-(hydroxymethyl)phenyl isobutyrate, particularly hydrates of Fesoterodine. “Fesoterodine” also includes pharmaceutically acceptable salts of 2-[(1R)-3-(diisopropylamino)-1-phenylpropyl]-4-(hydroxymethyl)phenyl isobutyrate, particularly the hydrogen fumarate salt, as well as the free base. It is also possible to use any salts and free base form of fesoterodine, including polymorphs, hydrates, solvates or amorphous forms.

The fesoterodine salt can be a salt of a polybasic acid. Examples may be chosen from the group of polybasic mineral acids, such as e.g. sulfuric acid and phosphoric acid, or of polybasic organic acids. Preferred examples are salts of di- or tricarboxylic acids such as fesoterodine maleate, fesoterodine oxalate, fesoterodine citrate, fesoterodine phthalate, fesoterodine fumarate, fesoterodine succinate, fesoterodine tartrate, fesoterodine malonate, fesoterodine malate, etc. In particular embodiments, the fesoterodine salt may be a partially hydrogenated di- or tricarboxylic acid salt, particularly a salt such as hydrogen fumarate or hydrogen maleate. A particularly preferred salt is fesoterodine hydrogen fumarate.

Fesoterodine or a pharmaceutically acceptable salt, preferably Fesoterodine hydrogen fumarate, or the free base, in the composition may be present in an amount of about 0.5-28 mg, or about 0.5-20 mg, preferably about 1-16 mg, about 1-12 mg, more preferably about 1-8 mg, and even more preferably about 2, about 4 or about 8 mg per dosage unit (based on the content of Fesoterodine or its salt, e.g., Fesoterodine hydrogen fumarate), or free base.

The term ‘sugar alcohols’ used hereinbefore and throughout the description hereinafter refers to polyhydric sugar and its derivatives, for example mannitol, xylitol, maltitol, isomaltitol, erythritol, lactitol and sorbitol.

The pharmaceutical composition described herein was found to retain at least 80% of potency of fesoterodine or salts thereof when stored at 40° C./75% R.H for 3 months and/or when stored at 50° C./80% R.H for at least 1 month. The amount of diol and diester impurity in the composition was found to be less than 2% and 1% by weight of fesoterodine or salt thereof respectively when determined after subjecting to storage in accelerated stability conditions.

The compositions of invention can be developed into dosage form to exhibit immediate release, extended release, sustained release, controlled release, modified release and delayed release or combination thereof. Such compositions can be prepared using rate controlling polymers. Preferably, the composition of the invention is in the form of an extended release composition.

The term “extended release” is used in its conventional sense to refer to a formulation that provides for gradual release of fesoterodine or salts thereof over an extended period of time, and that preferably, although not necessarily, results in substantially constant blood levels of fesoterodine are maintained within a therapeutic range but below toxic levels over an extended time period, preferably for at least about 4 hours, more preferably for at least about 6 hours after administration at steady-state.

The stable pharmaceutical composition of fesoterodine or salts thereof in 10, accordance with the present invention when developed in extended release dosage form, exhibits a dissolution profile which is suitable for once daily administration exhibiting a drug release of fesoterodine; its salt or active metabolite over the period of at least 12 hours.

In an embodiment, the stable pharmaceutical composition of fesoterodine or salts thereof provides extended release of fesoterodine, its salt or active metabolite thereof over the period of at least 24 hours.

In a preferred embodiment, the stable pharmaceutical composition exhibits a dissolution profile of fesoterodine or salts thereof in simulated gastric fluid (dissolution parameter-0.1N HCl, 900 ml water, 75 rpm, USP II type dissolution apparatus) such that at least 20% of fesoterodine is released in 4 hours, at least 40% of fesoterodine is released in 8 hours, at least 60% fesoterodine is released after 12 hours, and at least 70% fesoterodine is released in 24 hours.

Suitable “rate controlling polymers” may include one or more hydrophilic and hydrophobic polymers or mixtures thereof.

Suitable hydrophilic polymers may include one or more of cellulosic polymers/copolymers or its derivatives including methyl cellulose, hydroxypropyl methylcellulose, hydroxyethyl cellulose, hydroxypropyl cellulose, hydroxyethyl methylcellulose, carboxymethylcellulose, sodium carboxymethylcellulose; polyacrylates, methyl acrylates, polyethylene oxides, polyethylene glycols, chitosan, gums, starch derivatives, polyurethanes, galactomannans, polysaccharides, polyalcohols, acrylic acid or acrylamide derivatives and the like.

Suitable hydrophobic polymers include one or more of ethyl cellulose, glycerol palmitostearate, beeswax, glycowax, carnaubawax, hydrogenated vegetable oil, glycerol monostearate, stearylalcohol, glyceryl behenate, polyanhydrides, methyl acrylates and the like.

The stable composition of fesoterodine or salt thereof may be accomplished by homogeneously embedding drug containing rate-controlling hydrophilic polymers, being a soluble, partially soluble or insoluble network of viscous, hydrophilic polymers, held together by physical or chemical entanglements, by ionic or crystalline interactions, by complex formation, by hydrogen bonds or van der Waals forces. The hydrophilic matrix swells upon contact with water, thereby creating a protective gel layer from which the active ingredient is slowly, gradually, continuously released in time either by diffusion through the polymeric network, by erosion of the gel layer, by dissolution of the polymer, or by a combination of these release mechanisms.

The polymers used can also be eroding or non-eroding or combination of both. The polymers, which may be used for bioadhesion, are described below.

Natural polymers include but are not limited to proteins (e.g., hydrophilic proteins), such as pectin, zein, modified zein, casein, gelatin, gluten, serum albumin, or collagen, chitosan, oligosaccharides and polysaccharides such as cellulose, dextrans, tamarind seed polysaccharide, gellan, carrageenan, xanthan gum, gum Arabic; hyaluronic acid, polyhyaluronic acid, alginic acid, sodium alginate.

When the bioadhesive polymer is a synthetic polymer, the synthetic polymer is typically selected from but are not limited to polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric acid), poly(maleic acid), and blends and copolymers or mixtures thereof.

Other polymers suitable for use in the invention include, but are not limited to, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), polyvinyl acetate), polyvinyl chloride, polystyrene, polyvinyl pyrrolidone, polyvinylphenol, Polylactides, polyglycolides and copolymers thereof, poly(ethylene terephthalate), poly(butyric acid), poly(valeric acid), poly(lactide-co-caprolactone), poly[lactide-co-glycolide], polyanhydrides (e.g., poly(adipic anhydride)), polyorthoesters, blends and copolymers thereof.

The amount of rate-controlling polymer present in the pharmaceutical composition ranges from about 10% w/w to about 80% w/w of the composition.

In an embodiment, the rate-controlling polymer employed in the composition is water soluble polymer, preferably, hydroxypropyl methylcellulose (HPMC) and its various commercially available grades such as Methocel®. Preferably, rate-controlling polymers present in an amount that allows for the formation of a gel matrix, or swells upon contact with water and in order to provide gradual releasing of the active ingredient.

Suitable dosage form comprises one or more of tablets, multilayered tablets, capsules, pellets, granules, spheroids, beads, minitablets in capsule, pellets in capsule, granules in capsule, powder. Further the powder or granules can be suspended to give a pharmaceutically acceptable oral suspension.

In an embodiment, the composition of fesoterodine or its salt when devised to provide the extended release, it is preferably developed into dosage forms such as matrix-tablets/granules/pellets, coated tablets/granules/pellets or multiple unit particles which can be filled into capsules or compressed to form minitablets or tablets.

The pharmaceutical composition can also be given a film coating as necessary. Preferably, a moisture barrier film coating can be given in order to minimize the degradation of fesoterodine or salt thereof due to moisture. The moisture barrier film coating can be formed from commercially available products such as Colorcon's OPADRY®, for example OPADRY® II 85-series (a PVA-based coating) or OPADR Y® amb (Aqueous Moisture Barrier). The film coating preparation further may contain plasticizers.

Alternatively, the pharmaceutical composition of fesoterodine or its salt can be developed using various osmotic-controlled release oral systems (OROS) known in the art.

The invention further provides a process for preparing the stable pharmaceutical composition of fesoterodine or salts thereof.

The stable pharmaceutical compositions of fesoterodine or salt thereof may be prepared by processes known to the person having ordinary skill in the art of pharmaceutical technology such as direct compression, wet or dry granulation, slugging, hot melt granulation, hot melt extrusion, fluidized bed granulation, extrusion-spheronization, spray drying and solvent evaporation.

In an embodiment, the stable composition of fesoterodine or salt thereof is prepared by dry/wet granulating fesoterodine or salt thereof with one of more pharmaceutically acceptable excipients and then optionally mixing the granules with other excipients.

It has been surprisingly found that degradation of fesoterodine or salt thereof can be prevented if the composition is prepared by dry mixing and compressing the excipients. Optionally, the resultant composition may be pressed into tablets and coated.

Thus, in an embodiment, the stable composition of fesoterodine or salt thereof is prepared by dry mixing and compressing fesoterodine or salt thereof with one of more pharmaceutically acceptable excipients. The pharmaceutical composition made from this process was found to have at least 80%, preferably 90% of the original amount of fesoterodine or salt thereof remaining in undegraded form.

The stable composition of the present invention can be prepared in the form of mixture, granules, pellets, coating dispersion by various methods known to the person skilled in the art.

The invention further provides process of preparing stable pharmaceutical composition of fesoterodine or salts thereof which provides extended release of fesoterodine, salt and active metabolite thereof. The composition may be developed using methods known to those skilled in the art. Such composition comprises particles, agglomerates, granules, pellets, microspheres, liposomes, sphericles, minitablets, microcapsules, tablets, cores of fesoterodine or salts thereof, mixed or coated with one or more rate-controlling polymer(s).

In an embodiment, the extended release composition is prepared by mixing fesoterodine or salts thereof with one or more pharmaceutically acceptable rate-controlling polymers. The mixture can be blended with other pharmaceutically acceptable excipients, lubricated and formulated into suitable dosage form.

In a still another embodiment, the stable compositions may be prepared by mixing and granulating fesoterodine or salts thereof with one or more rate-controlling polymers to form granules. The granules can be mixed with other pharmaceutically acceptable excipients, lubricated and formulated into suitable dosage form. Further, the dosage form can be coated with film-forming polymers.

In order to extend the shelf life, the composition in accordance with the present invention can be stored under ambient dry conditions, for example in presence of a desiccant, such as silica, typically encased in a pack suitable for absorption of moisture.

The pharmaceutically acceptable excipients may include one or more binders, fillers, lubricants, disintegrants, glidants and the like.

Suitable fillers may include one or more of microcrystalline cellulose, starch, dibasic calcium phosphate, tribasic calcium phosphate, calcium carbonate, dextrose, kaolin, magnesium carbonate, magnesium oxide; sugars such as lactose or sucrose; and the like. The amount of filler present in present in the pharmaceutical composition ranges from about 1% to about 80% w/w of the composition.

Suitable disintegrants may include one or more of croscarmellose sodium, sodium starch glycolate, pregelatinized starch, sodium carboxymethyl cellulose, cross-linked polyvinylpyrrolidone and the like. The amount of disintegrant present in the pharmaceutical composition ranges from about 1% to about 15% w/w of the composition.

Suitable binders may include one or more of hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, pregelatinized starch, sodium alginate, gums, synthetic resins and the like. The amount of binder present in the pharmaceutical composition ranges from about 0.5% to about 10% w/w of the composition.

Suitable lubricants and glidants may include one or more of talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and the like. The amount of lubricant or glidant present in the pharmaceutical composition ranges from about 0.1% to about 5% w/w of the composition.

The invention also provides a method of treating patients suffering from overactive bladder and having symptoms such as urinary incontinence, specifically urinary urge incontinence, urinary urgency and/or urinary frequency by administering a therapeutically effective amount of any of the compositions as described herein. In particular, disclosed is a method of treating patients suffering from overactive bladder that may have symptoms such as urinary incontinence, urinary urge incontinence, urinary urgency and/or urinary frequency by administering a unit dosage form of the Fesoterodine compositions described herein.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Example 1

TABLE 1

| S.N. | Ingredient | (% w/w) |
|---|---|---|
| 1 | Fesoterodine fumarate | 0.1-10% |
| 2 | Polyvinyl pyrrolidone | 1-5% |
| 3 | Microcrystalline cellulose | 5-45% |
| 4 | Hydroxypropyl methylcellulose | 10-60% |
| 5 | Colloidal silicone dioxide | 0.2-3% |
| 6 | Magnesium Stearate | 0.1-3% |

### Procedure:

Fesoterodine, polyvinyl pyrrolidone and microcrystalline cellulose were mixed and granulated with hydro alcoholic granulating solvent. Granules were dried and mixed with hydroxypropyl methylcellulose and colloidal silicon dioxide. The granules were lubricated with magnesium stearate and compressed into tablets using suitable tooling.

### Example 2

TABLE 2

| S.N. | Ingredient | (% w/w) |
|---|---|---|
| 1 | Fesoterodine fumarate | 0.1-10% |
| 2 | Polyvinyl pyrrolidone | 1-5% |
| 3 | Microcrystalline cellulose | 5-45% |
| 4 | Polyethylene oxide | 10-60% |
| 5 | Colloidal silicone dioxide | 0.2-3% |
| 6 | Magnesium Stearate | 0.1-3% |

### Procedure:

Fesoterodine, polyvinyl pyrrolidone and microcrystalline cellulose were mixed and granulated with hydro alcoholic granulating solvent. Granules were dried and mixed with polyethylene oxide and colloidal silicon dioxide. The granules were lubricated with magnesium stearate and compressed into tablets using suitable tooling.

### Example 3

TABLE 3

| S.N. | Ingredient | (% w/w) |
|---|---|---|
| 1 | Fesoterodine fumarate | 0.1-10% |
| 2 | Polyvinyl pyrrolidone | 1-5% |
| 3 | Microcrystalline cellulose | 5-45% |
| 4 | Lactose | 5-20% |
| 5 | Hydroxypropyl methylcellulose | 10-60% |
| 6 | Colloidal silicone dioxide | 0.2-3% |
| 7 | Magnesium Stearate | 0.1-3% |

### Procedure:

Fesoterodine, polyvinyl pyrrolidone and microcrystalline cellulose were mixed and granulated with hydro alcoholic granulating solvent. Granules were dried and mixed with lactose, hydroxypropyl methylcellulose and colloidal silicon dioxide. The granules were lubricated with magnesium stearate and compressed into tablets using suitable tooling.

### Example 4

TABLE 4

| S.N. | Ingredient | (% w/w) |
|---|---|---|
| 1 | Fesoterodine fumarate | 0.1-10% |
| 2 | Hypromellose | 2-15% |
| 3 | Microcrystalline cellulose | 5-45% |
| 4 | Lactose | 5-20% |
| 5 | Hydroxypropyl methylcellulose | 10-60% |
| 6 | Polyethylene Glycol | 1-10% |
| 7 | Colloidal silicone dioxide | 0.2-3% |
| 8 | Magnesium Stearate | 0.1-3% |

### Procedure:

Fesoterodine was granulated with aqueous solution of hypromellose. Granules were dried and mixed with microcrystalline cellulose, lactose, hypromellose, polyethylene glycol and colloidal silicon dioxide. The granules were lubricated with magnesium stearate and compressed into tablets using suitable tooling.

### Example 5

TABLE 5

| S.N. | Ingredient | Mg/Tablet |
|---|---|---|
| 1 | Fesoterodine fumarate | 8.0 |
| 2 | Microcrystalline cellulose | 90.0 |
| 3 | Corn Starch | 15.0 |
| 4 | Lactose monohydrate | 80.5 |
| 5 | Hydroxypropyl methylcellulose | 140.0 |
| 6 | Povidone K 30 | 8.0 |
| 7 | Talc | 3.5 |
| 8 | Magnesium Stearate | 5.0 |
| 9 | Opadry ® AMB yellow | 7.0 |

### Procedure:

Fesoterodine, microcrystalline cellulose, corn starch, lactose monohydrate, hydroxypropyl methylcellulose and povidone were mixed to form a drug-excipient blend. The blend was then lubricated with talc and magnesium stearate and compressed into tablets using suitable tooling. The compressed tablets were then coated with Opadry® AMB yellow ready mix.

### Example 6

### In Vitro Dissolution Profile of Toviaz® Tablet and Formulation of the Invention

TABLE 6

| | | Formulation of |
|---|---|---|
| | Toviaz ® | the Invention |
| Time (hr) | (% Drug release) | (% Drug release) |
| 0.5 | 8.7 | 8.9 |
| 1 | 14.8 | 13.8 |
| 2 | 25.0 | 21.9 |
| 4 | 38.5 | 34.4 |
| 8 | 57.9 | 53.4 |
| 10 | 63.9 | 60.9 |
| 12 | 68.6 | 66.8 |
| 16 | 73.9 | 76.5 |
| 20 | 75.7 | 80.8 |
| 24 | 75.0 | 82.6 |

Dissolution parameters—USP Type II apparatus (75 rpm) was used wherein 900 ml of water and 0.1N HCl was used as medium.

TABLE 7

| | | Formulation of |
|---|---|---|
| | Toviaz ® | the Invention |
| Time (hr) | (% Drug release) | (% Drug release) |
| 0.5 | 8.3 | 9.2 |
| 1 | 14.8 | 14.5 |
| 2 | 24.7 | 22.7 |
| 4 | 39.4 | 35.6 |
| 8 | 59.7 | 54.4 |
| 10 | 66.6 | 62.2 |
| 12 | 71.9 | 68.8 |
| 16 | 78.8 | 79.0 |
| 20 | 82.2 | 85.6 |
| 24 | 83.3 | 90.5 |

Dissolution parameters—USP Type II apparatus (75 rpm) was used wherein phosphate buffer of pH 6.8 was used as medium.

The dissolution data indicates that the rate and extent of drug release from the formulation of the present invention is relatively equivalent to that of marketed formulation (Toviaz®).

### Example 6

### Stability Study

The accelerated stability study of the formulation of the invention was conducted at 50° C./80% R.H. over the period of 1 month.

The amount of the impurities measured in the formulation after the storage period indicates that the formulation of the invention is stable under stress conditions.

TABLE 8

| | | Stored in HDPE | | Stored in HDPE container | |
|---|---|---|---|---|---|
| | | container containing | | containing 2 gm silica gel | |
| | | 2 gm silica gel | | & 1 gm molecular sieve | |
| Impurity | Initial | 2 Week | 1 Month | 2 Week | 1 Month |
| Diol | 0.03 | 0.61 | 1.3 | 0.21 | 0.31 |
| Diester | 0.04 | 0.24 | 0.48 | 0.11 | 0.15 |
| RRT 1.20 | ND | ND | ND | ND | ND |
| (known) | | | | | |
| RRT 1.37 | 0.06 | 0.05 | 0.14 | 0.05 | 0.06 |
| (known) | | | | | |
| RRT 1.70 | ND | ND | ND | ND | ND |
| (known) | | | | | |
| RRT 0.17 | ND | 0.11 | BQL | 0.06 | 0.03 |
| RRT 0.39 | ND | 0.03 | BQL | 0.03 | 0.03 |
| RRT 0.53 | ND | 0.03 | 0.08 | ND | ND |
| RRT 0.72 | ND | 0.04 | 0.12 | 0.03 | 0.05 |
| RRT 0.77 | ND | ND | 0.07 | ND | ND |
| Maximum | ND | ND | 0.11 | 0.12 | 0.06 |
| unknown | | | | | |

The amount of impurity refers to the percent by weight of fesoterodine or salt thereof.

1. A stable pharmaceutical composition comprising fesoterodine or salts thereof, one or more rate-controlling polymers and one or more pharmaceutically acceptable excipients, characterized in that the composition is free of sugar alcohol.

2. The stable pharmaceutical composition of claim 1, wherein the rate-controlling polymer comprises one or more of hydrophilic polymers, hydrophobic polymers, or combinations thereof.

3. The stable pharmaceutical composition of claim 2, wherein the hydrophilic polymer comprises one or more of methyl cellulose, hydroxypropyl methylcellulose, hydroxyethyl cellulose, hydroxypropyl cellulose, hydroxyethyl methylcellulose, carboxymethylcellulose, sodium carboxymethylcellulose; polyacrylates, methyl acrylates, polyethylene oxides, polyethylene glycols, chitosan, gums, starch derivatives, polyurethanes, galactomannans, polysaccharides, and polyalcohols.

4. The stable pharmaceutical composition of claim 1, wherein the amount of rate-controlling polymer ranges from about 10% w/w to about 80% w/w of the composition.

5. The stable pharmaceutical composition of claim 1, wherein the composition is in the form of an immediate release, extended release, sustained release, controlled release, modified release, or a delayed release dosage form.

6. The stable pharmaceutical composition of claim 5, wherein the composition is in the form of an extended release dosage form.

7. The stable pharmaceutical composition of claim 6, wherein the extended release dosage form comprises a matrix of fesoterodine or a salt thereof and one or more rate controlling polymers.

8. The stable pharmaceutical composition of claim 7, wherein the extended release dosage form comprises multiple-unit particles comprising fesoterodine or salts thereof mixed or coated with one or more rate-controlling polymers.

9. The stable pharmaceutical composition of claim 1, wherein the composition retains at least 80% of potency of fesoterodine or salts thereof after storage for three months at 40° C. and 75% relative humidity.

10. The stable pharmaceutical composition of claim 1, wherein the composition retains at least 80% of potency of fesoterodine or salts thereof after storage for one month at 50° C. and 80% relative humidity.

11. The stable pharmaceutical composition of claim 1, wherein the composition contains less than 2.0% w/w of diol impurity relative to the total weight of fesoterodine or salts thereof after storage for one month at 50° C. and 80% relative humidity.

12. The stable pharmaceutical composition of claim 1, wherein the composition contains less than 1.5% w/w of diol impurity relative to the total weight of fesoterodine or salts thereof after storage for one month at 50° C. and 80% relative humidity.

13. The stable pharmaceutical composition of claim 1, wherein the composition contains less than 1.0% w/w of diester impurity relative to the total weight of fesoterodine or salt thereof after storage for one month at 50° C. and 80% relative humidity.

14. The stable pharmaceutical composition of claim 1, wherein the composition contains less than 0.5% w/w of diester impurity relative to the total weight of fesoterodine or salt thereof after storage for one month at 50° C. and 80% relative humidity.

15. The stable pharmaceutical composition of claim 1, wherein the fesoterodine salt is fesoterodine fumarate.

16. The stable pharmaceutical composition of claim 1, wherein one or more pharmaceutically acceptable excipients comprise one or more binders, fillers, lubricants, disintegrants, glidants, or combinations thereof.

17. The stable pharmaceutical composition of claim 1, wherein the composition exhibits a dissolution profile of fesoterodine or salts thereof in simulated gastric fluid such that at least 30% of fesoterodine is released in 4 hours, at least 40% of fesoterodine is released in 8 hours, at least 60% fesoterodine is released in 12 hours, and at least 70% fesoterodine is released in 24 hours.

18. A stable pharmaceutical composition comprising:
(a) about 0.1-10.0% w/w of fesoterodine fumarate;
(b) about 1.0-5.0% w/w of polyvinyl pyrrolidone;
(c) about 5.0-45.0% w/w of microcrystalline cellulose;
(d) about 10.0-60.0%) w/w of hydroxypropyl methycellulose;
(e) about 0.1-0.3% w/w of one or more of talc, magnesium stearate and colloidal silicone dioxide; and
characterized in that the composition is free of sugar alcohol.

19. The stable pharmaceutical composition of claim 18, wherein the composition further comprises about 5.0% to about 20% w/w of lactose.

20. The stable pharmaceutical composition of claim 18, wherein the composition further comprises about 1.0% to about 10.0% w/w of polyethylene glycol.

21. The stable pharmaceutical composition of claim 21, wherein the composition further comprises about 3.0% to about 20.0% w/w of starch.

22. A process for the preparation of a stable pharmaceutical composition comprising fesoterodine or salts thereof, the process comprising dry or wet granulating fesoterodine or a salt thereof with one or more rate controlling polymers and one or more pharmaceutical excipients.

23. A process for the preparation of a stable pharmaceutical composition comprising fesoterodine or salts thereof, the process comprising mixing and compressing fesoterodine or salts thereof with one or more rate-controlling polymers optionally, with one or more pharmaceutically acceptable excipients, wherein the composition is free of sugar alcohol.

24. A process according to claim 23, for the preparation of a stable pharmaceutical composition of fesoterodine or salts thereof, the process comprising:
(a) mixing fesoterodine or salts thereof, one or more rate-controlling polymers and more pharmaceutically acceptable excipients;
(b) granulating the mixture to form granules; and
(c) converting the granules prepared in step (b) into a suitable dosage form.

25. A method of treating a patient suffering from overactive bladder by administering a therapeutically effective amount of a stable pharmaceutical composition comprising fesoterodine or salts thereof, one or more rate-controlling polymers and one or more pharmaceutically acceptable excipients, characterized in that the composition is free of sugar alcohol.
